(19) United States
(12) Patent Application Publication
(10) Pub. No.: US 2021/0285138 A1
(43) Pub. Date: Sep. 16, 2021

(54) FIBER MESH SHEET, METHOD FOR MANUFACTURING FIBER MESH SHEET, AND CELL CULTURE CHIP FORMED OF FIBER MESH SHEET

(71) Applicant: Panasonic Intellectual Property Management Co., Ltd., Osaka (JP)

(72) Inventors: TAICHI NAKAMURA, Osaka (JP); NORIHITO TSUKAHARA, Kyoto (JP); KOUJI IKEDA, Hyogo (JP); KIYOTAKA TSUJI, Osaka (JP)

(21) Appl. No.: 17/198,185

(22) Filed: Mar. 10, 2021

(30) Foreign Application Priority Data
Mar. 16, 2020 (JP) 2020-045525
Feb. 17, 2021 (JP) 2021-023538

(51) Int. Cl. D04H 1/74; C12N 5/00; D04H 1/541

(52) U.S. Cl. CPC D04H 1/74; D04H 1/541; C12N 5/0068

(57) ABSTRACT

A fiber mesh sheet is provided, in which the fiber mesh sheet has a mesh structure in which two or more layers of planar fiber arrangement groups are laminated, where, in each of the planar fiber arrangement groups, longitudinal directions of a plurality of fibers made of a polymer material are arranged in a plane along one direction, longitudinal directions of the fibers in one of the fiber arrangement groups intersect those of the fibers in the other fiber arrangement group in two adjacent layers of the fiber arrangement groups at an intersecting angle of 30° or more and 150° or less in a plan view seen from a direction perpendicular to the plane, an upper part of a cross section of the fiber in the fiber arrangement group at a lowermost layer is a substantially circular shape, and a lower part of a cross section of the fiber in the fiber arrangement group at the lowermost layer is a substantially flat shape, where the upper part is a side on which the adjacent fiber arrangement group is present, and the lower part is a side on which the adjacent fiber arrangement group is not present, and a cross section of the fiber in the fiber arrangement group at a layer other than the lowermost layer is a substantially circular shape.

### BACKGROUND

### 1. Technical Field

The present disclosure relates to a fiber mesh sheet, a method for manufacturing a fiber mesh sheet, and a cell culture chip formed of the fiber mesh sheet.

### 2. Description of the Related Art

In recent years, an organ-on-a-chip (OoC) has been actively developed as a chip used for cell culture (refer to, for example, Japanese Patent Unexamined Publication No. 2019-180354). An OoC is a cell culture chip that reproduces tissue functions of a living body on a microscale by culturing cells in an artificial microspace obtained by combining glass, resin, and the like.

By administering a medicine to cells cultured using such a cell culture chip, it is possible to evaluate efficacy of medicines, toxicity tests, and tests for absorption, metabolism, excretion, and the like, which were conventionally evaluated by animal tests using mice, in an in vitro artificial chip using cells having a function closer to that of a living body.

### SUMMARY

In a fiber mesh sheet according to one aspect of the present disclosure, the fiber mesh sheet has a mesh structure in which two or more layers of planar fiber arrangement groups are laminated, where, in each of the planar fiber arrangement groups, longitudinal directions of a plurality of fibers made of a polymer material are arranged in a plane along one direction,
longitudinal directions of the fibers in one of the fiber arrangement groups intersect those of the fibers in the other fiber arrangement group in two adjacent layers of the fiber arrangement groups at an intersecting angle of 30° or more and 150° or less in a plan view seen from a direction perpendicular to the plane,
an upper part of a cross section of the fiber in the fiber arrangement group at a lowermost layer is a substantially circular shape, and a lower part of a cross section of the fiber in the fiber arrangement group at the lowermost layer is a substantially flat shape, where the upper part is a side on which the adjacent fiber arrangement group is present, and the lower part is a side on which the adjacent fiber arrangement group is not present, and
a cross section of the fiber in the fiber arrangement group at a layer other than the lowermost layer is a substantially circular shape.

In a method for manufacturing a fiber mesh sheet according to one aspect of the present disclosure, the method including: a step of laminating two or more layers of planar fiber arrangement groups, where, in each of the planar fiber arrangement groups, longitudinal directions of a plurality of fibers made of a polymer material are arranged in a plane along one direction, such that longitudinal directions of the fibers in one of the fiber arrangement groups intersect those of the fibers in the other fiber arrangement group in two adjacent layers of the fiber arrangement groups at an intersecting angle of 30° or more and 150° or less in a plan view seen from a direction perpendicular to the plane; and
a step of performing a heat treatment at a temperature equal to or higher than a melting point of the polymer material of the fiber and lower than a temperature that the fiber is melted and cut, in which the two adjacent layers of the fiber arrangement groups are intertwined by the step of performing the heat treatment at a majority of portions at which the two adjacent layers are in contact with each other.

### BRIEF DESCRIPTION OF THE DRAWINGS

FIG. 1A is a schematic perspective view showing a configuration of a fiber mesh sheet according to a first exemplary embodiment;

FIG. 1B is a schematic perspective view showing a structure and dimensions of each of fibers constituting a first layer and a second layer of the fiber mesh sheet of FIG. 1A;

FIG. 2 is a flowchart of a method for manufacturing the fiber mesh sheet according to the first exemplary embodiment;

FIG. 3 is an exploded perspective view showing a configuration of a cell culture chip according to the first exemplary embodiment; and

FIG. 4 is Table 1 showing conditions of each of comparative examples and examples and culture results in a case where the cell culture chip was used.

### DETAILED DESCRIPTIONS

In the conventional cell culture chip disclosed in Japanese Patent

Unexamined Publication No. 2019-180354, for example, a micromesh sheet as a scaffold for culturing cells is used to provide a cell sheet containing hepatocytes or intestinal cells having a function closer to that of a living body.

In some cases, two types of cells are formed by culturing cells independently in upper and lower cell sheets, and different fluids are perfused independently in the upper and lower sheets to evaluate permeation of drugs through the cell sheets. In this case, it is required to set a size of a mesh opening to a size that inhibits passage of one cell so that cells in the upper and lower sheets are cultured while being separated. This leads to a problem in which the mesh opening is easily clogged with a test substance, and drug permeability is hindered.

In particular, this problem has become more apparent because a molecular weight has been increasing in candidate compounds for latest new drugs.

Accordingly, the present disclosure solves the above-mentioned conventional problems, and an object thereof is to provide a fiber mesh sheet used for a cell culture chip in which two types of cells can be cultured while being separated into upper and lower parts even when the fiber mesh sheet has a mesh opening having an appropriate size capable of inhibiting clogging of the mesh opening with a test substance.

In a fiber mesh sheet according to a first aspect, the fiber mesh sheet has a mesh structure in which two or more layers of planar fiber arrangement groups are laminated, where, in each of the planar fiber arrangement groups, longitudinal directions of a plurality of fibers made of a polymer material are arranged in a plane along one direction,
longitudinal directions of the fibers in one of the fiber arrangement groups intersect those of the fibers in the other fiber arrangement group in two adjacent layers of the fiber arrangement groups at an intersecting angle of 30° or more and 150° or less in a plan view seen from a direction perpendicular to the plane,
an upper part of a cross section of the fiber in the fiber arrangement group at a lowermost layer is a substantially circular shape, and a lower part of a cross section of the fiber in the fiber arrangement group at the lowermost layer is a substantially flat shape, where the upper part is a side on which the adjacent fiber arrangement group is present, and the lower part is a side on which the adjacent fiber arrangement group is not present, and
a cross section of the fiber in the fiber arrangement group at a layer other than the lowermost layer is a substantially circular shape.

In the fiber mesh sheet according to a second aspect, in the first aspect, a contact angle between the substantially circular shape in the fiber arrangement group at the layer other than the lowermost layer and water may be 90° or more and 150° or less.

In the fiber mesh sheet according to a third aspect, in the first or second aspect, an average diameter of the fibers in the fiber arrangement group may be 1 μm or more and 50 μm or less.

In a method for manufacturing a fiber mesh sheet according to a fourth aspect, the method including: a step of laminating two or more layers of planar fiber arrangement groups, where, in each of the planar fiber arrangement groups, longitudinal directions of a plurality of fibers made of a polymer material are arranged in a plane along one direction, such that longitudinal directions of the fibers in one of the fiber arrangement groups intersect those of the fibers in the other fiber arrangement group in two adjacent layers of the fiber arrangement groups at an intersecting angle of 30° or more and 150° or less in a plan view seen from a direction perpendicular to the plane; and
a step of performing a heat treatment at a temperature equal to or higher than a melting point of the polymer material of the fiber and lower than a temperature that the fiber is melted and cut,
in which the two adjacent layers of the fiber arrangement groups are intertwined by the step of performing the heat treatment at a majority of portions at which the two adjacent layers are in contact with each other.

In the method for manufacturing a fiber mesh sheet according to a fifth aspect, in the fourth aspect, a lower part of a cross section of the fiber in the fiber arrangement group at a lowermost layer may be formed into a substantially flat shape by the step of performing the heat treatment at the temperature equal to or higher than the melting point of the polymer material of the fiber and lower than the temperature that the fiber is melted and cut, where the lower part is a side on which the adjacent fiber arrangement group is not present.

A cell culture chip according to a sixth aspect includes the fiber mesh sheet according to any one of the first to third aspects.

As described above, according to the fiber mesh sheet according to the present disclosure, it is possible to separate, capture, and recover a substance having a size of one cell in the lower part having a substantially flat shape of the fibers in the fiber arrangement group at the lowermost layer.

Furthermore, by using this fiber mesh sheet as a scaffold for culturing cells to configure the cell culture chip, two types of cells can be cultured while being separated into upper and lower parts even when the fiber mesh sheet has a mesh opening having an appropriate size capable of inhibiting clogging of the mesh opening with a test substance. Accordingly, it is possible to cope with evaluation of candidate compounds for latest new drugs in which a molecular weight has been increasing.

Hereinafter, a fiber mesh sheet according to an exemplary embodiment, a method for manufacturing a fiber mesh sheet according to an exemplary embodiment, and a cell culture chip according to an exemplary embodiment will be described with reference to attached drawings. In the drawings, substantially the same members are designated by the same reference numerals.

### First Exemplary Embodiment

### Fiber Mesh Sheet

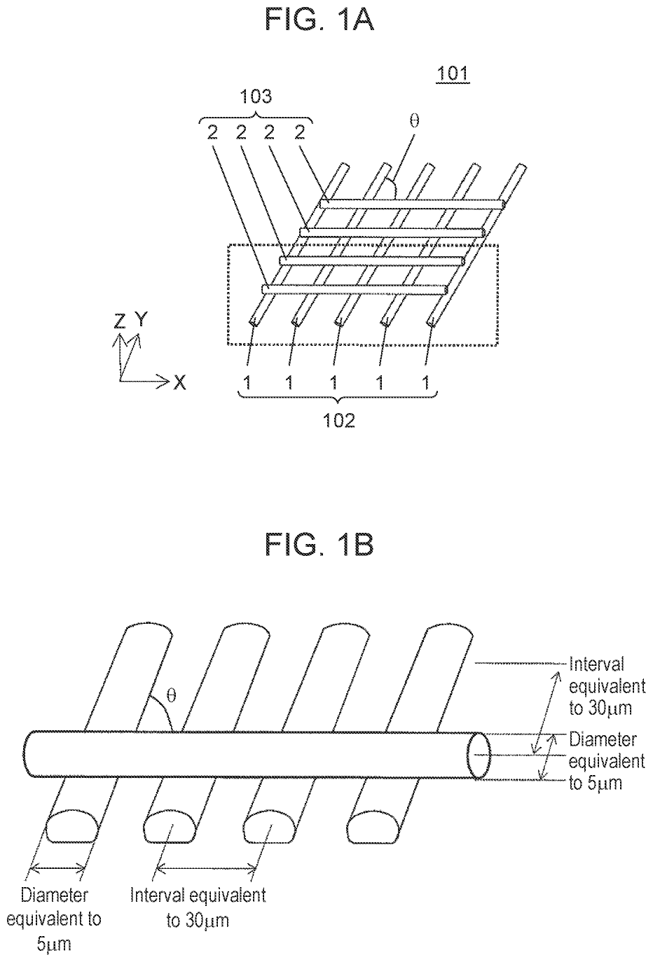

FIG. 1A is a schematic perspective view showing a configuration of fiber mesh sheet 101 according to a first exemplary embodiment. FIG. 1B is a schematic perspective view showing a structure and dimensions of each of fibers constituting a first layer and a second layer of the fiber mesh sheet of FIG. 1A. In the drawings, for convenience, an in-plane of fiber arrangement group 102 at a first layer and fiber arrangement group 103 at a second layer is shown as an XY plane, and a lamination direction is shown as a Z direction.

Fiber mesh sheet 101 has a mesh structure in which two or more layers of planar fiber arrangement groups 102 and 103 are laminated, where, in each of the planar fiber arrangement groups, longitudinal directions of a plurality of fibers 1 and 2 made of a polymer material are arranged in a plane (XY plane) along one direction. Longitudinal directions of fibers 1 in fiber arrangement group 102 intersect those of fibers 2 in fiber arrangement group 103 in two adjacent layers of fiber arrangement groups 102 and 103 at an intersecting angle θ of 30° or more and 150° or less in a plan view seen from a direction (Z direction) perpendicular to the plane. Fiber arrangement group 102 of the first layer is a fiber group in which longitudinal directions of a plurality of fibers 1 made of a polymer material are aligned in one direction in fine lines at equal intervals, and an upper part of a cross-sectional shape of each of fibers 1 is a substantially circular shape, and a lower part of a cross-sectional shape of each of fibers 1 is a substantially flat shape. The upper part is a side on which the adjacent fiber arrangement group is present, that is, a forward direction in the Z direction. The lower part is a side on which the adjacent fiber arrangement group is not present, that is, a backward direction in the Z direction. Fiber arrangement group 103 of the second layer is a fiber group in which longitudinal directions of a plurality of fibers 2 made of a polymer material are aligned in one direction in fine lines at equal intervals, and a cross-sectional shape of each of fibers 2 constituting fiber arrangement group 103 of the second layer is a substantially circular shape. Majority portions of the lower part of fibers 2 in fiber arrangement group 103 of the second layer are intertwined with and joined to the upper part of fibers 1 in fiber arrangement group 102 of the first layer. The term “intertwine” means that fiber 1 and fiber 2 intersect, and a part of each of fiber 1 and fiber 2 is joined at a position at which they are in contact with each other.

According to this fiber mesh sheet, longitudinal directions of fibers 1 in fiber arrangement group 102 intersect those of fibers 2 in fiber arrangement group 103 in two adjacent layers of fiber arrangement groups 102 and 103 at an intersecting angle θ of 30° or more and 150° or less in a plan view seen from a direction (Z direction) perpendicular to the plane. Accordingly, it is possible to inhibit both passage of cells and clogging with a test substance.

Furthermore, a contact angle between the substantially circular shape of fiber 2 in fiber arrangement group 103 of the second layer, which is not a lowermost layer, and water may be 90° or more and 150° or less. Accordingly, it can be further expected that a cell sheet having a function closer to that of a living body can be obtained by wetting in a lateral direction of the substantially circular shape of fiber 2 to bring water into contact with a cell type through a mesh opening.

Furthermore, an average diameter of fibers 1 and fibers 2 in fiber arrangement group 102 of the first layer and fiber arrangement group 103 of the second layer may be, for example, 1 μm or more and 50 μm or less.

The average diameter is an average value of diameters of fibers 1 and 2. A diameter of each of fibers 1 and 2 is a diameter of a cross section perpendicular to a length direction of each of the fibers. In a case where such a cross section is not circular, a maximum diameter may be regarded as a diameter. Furthermore, a width in a direction perpendicular to the length direction of the fiber when seen from a normal direction of one main surface of each of fiber arrangement group 102 of the first layer and fiber arrangement group 103 of the second layer may be regarded as a diameter of each of the fibers. An average fiber diameter is, for example, an average value obtained by image processing measurement of an average value of diameters of any position at ten fibers included in fiber arrangement group 102 of the first layer and fiber arrangement group 103 of the second layer.

In addition, a fiber arrangement group as a third or higher layer may be provided on fiber arrangement group 103 of the second layer, that is, in the forward direction of the Z direction.

### Method for Manufacturing Fiber Mesh Sheet

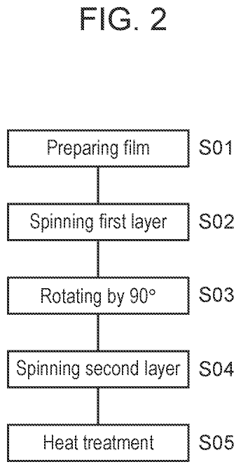

FIG. 2 is a flowchart of a method for manufacturing fiber mesh sheet 101 according to the first exemplary embodiment.

(1) S01 is a step of preparing a film. It is desirable that a surface of the film have appropriate peelability by being subjected to a fluorine treatment or the like. The reason for this is because an adhesive function to fibers is required when spinning the fibers on the film in each of steps S02 and S04 to be described later, and a function of peeling off the fiber mesh sheet from the film is required when the fiber mesh sheet is incorporated into a cell culture chip in a subsequent step.

(2) S02 is a step of spinning fiber arrangement group 102 of the first layer. A solution obtained by melting a polymer material used as fiber mesh sheet 101 through heating or a solution swollen with an organic solvent is applied to the film prepared in the step S01 in the same direction at equal intervals in fine lines.

The polymer material supplied in the molten state or the solution state is naturally cooled or naturally dried to form fibers only in a solid state.

In the first exemplary embodiment, polystyrene having low cytotoxicity is used as the polymer material, and a solution obtained by swelling pelletized polystyrene in DMF (N,N-dimethylformamide) as an organic solvent by 30% by weight is used and applied to fibers each having a diameter equivalent to 5 μm in the same direction at intervals equivalent to 30 μm. An average diameter of the fibers may be 1 μm or more and 50 μm or less.

(3) S03 is a step of rotating the film in which fiber arrangement group 102 of the first layer is spun in the step S02 by 90° in a plane.

(4) S04 is a step of spinning fiber arrangement group 103 of the second layer on the film rotated by 90° in the plane in the step S03. A polymer material used as fiber mesh sheet 101 is melted by heating or a solution swollen with an organic solvent is applied to the film prepared in the step S03 in the same direction at equal intervals in fine lines.

In the first exemplary embodiment, polystyrene having low cytotoxicity is used as the polymer material as in the step S02, and a solution obtained by swelling pelletized polystyrene in DMF (N,N-dimethylformamide) as an organic solvent by 30% by weight is used and applied to fibers each having a diameter equivalent to 5 μm in the same direction at intervals equivalent to 30 μm.

(5) S05 is a step of heating the fiber mesh sheet on the film created up to the step S04. Specifically, by heating, for a certain period of time, the polymer material (polystyrene in the first exemplary embodiment) at a temperature equal to or higher than a melting point and lower than a temperature that the fiber is melted and cut, a majority of portions, at which the upper part of fiber arrangement group 102 of the first layer and the lower part of fiber arrangement group 103 of the second layer are in contact with each other, are intertwined, and the lower part of fiber arrangement group 102 of the first layer becomes a substantially flat shape. Intertwining portions, at which the upper part of fiber arrangement group 102 of the first layer and the lower part of fiber arrangement group 103 of the second layer are in contact with each other, may be, for example, joining by melting intersecting fibers. Here, the temperature that the fiber is melted and cut is, for example, a temperature 100° C. higher than the melting point of the polymer material of the fiber.

According to the above descriptions, the fiber mesh sheet can be obtained.

### Cell Culture Chip

Hereinafter, cell culture chip 300 formed of fiber mesh sheet 101 according to the first exemplary embodiment will be further described.

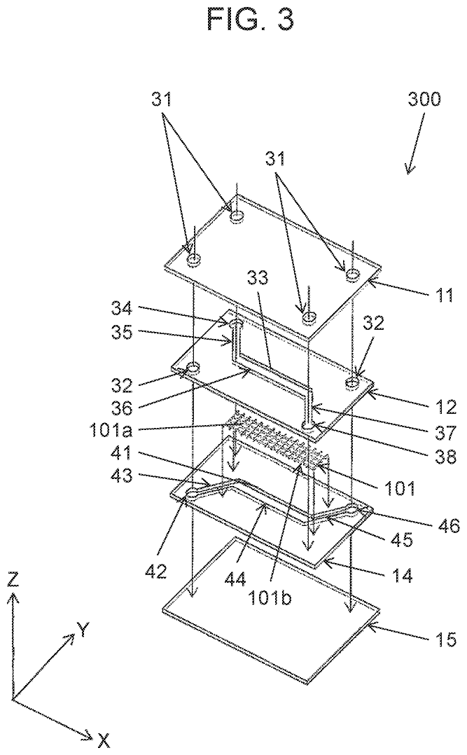

FIG. 3 is an exploded perspective view showing a configuration of cell culture chip 300 according to the first exemplary embodiment. In the drawing, for convenience, an in-plane of first board 11 and the like is shown as an XY plane, and a direction perpendicular to this is shown as a Z direction.

Cell culture chip 300 according to the first exemplary embodiment includes a main body, and fiber mesh sheet 101 manufactured by the method for manufacturing a fiber mesh sheet. The main body has a lamination structure in which first board 11, first partition layer 12, second partition layer 14, and second board 15, all of which have a main surface parallel to the XY plane, are laminated in this order along a predetermined direction (Z-axis direction in the drawing). Furthermore, fiber mesh sheet 101 is sandwiched by first partition layer 12 and second partition layer 14 of the main body.

The members constituting cell culture chip 300 will be described below.

### First Board

First board 11 is a plate-shaped member formed of a material such as glass. A material of first board 11 is not limited to glass, and any material such as resin and ceramics may be used. Furthermore, first board 11 is formed of a material having no cytotoxicity because first board 11 comes into contact with cells when culturing the cells.

In the first exemplary embodiment, first board 11 is a plate-shaped member having a rectangular main surface. Furthermore, first board 11 has holes 31 that penetrates first board 11 along a predetermined direction to lead to first partition layer 12 on which first board 11 is to be laminated.

### First Partition Layer

First board 11 may be configured to directly communicate with first partition layer 12, not via holes 31 of first board 11, in a case where a portion of first partition layer 12 is exposed in a manner of not overlapping with first board 11. First partition layer 12 is a plate-shaped member formed of a silicone resin.

First partition layer 12 has a first through-hole, and at least part of the first through-hole penetrates first board 11 to first partition layer 12 in a thickness direction (Z-axis direction). Details will be described later, but the first through-hole corresponds to first flow path 33.

Both ends of the first through-hole correspond to two of holes 31 formed in first board 11. Furthermore, first partition layer 12 has holes 32 which correspond to two holes 31 other than two holes 31 corresponding to the first through-hole, and which penetrate first partition layer 12 in the thickness direction to lead to second partition layer 14 on which first partition layer 12 is to be laminated.

### Fiber Mesh Sheet

Fiber mesh sheet 101 has first main surface 101a on first partition layer 12 side and second main surface 101b on second partition layer 14 side. In first main surface 101a, a cross-sectional shape of a fiber in the fiber arrangement group of the second layer in FIG. 1A is a substantially circular shape. In second main surface 101b, a cross-sectional shape of a fiber in the fiber arrangement group of the first layer in FIG. 1A is a flat shape. Furthermore, predetermined openings are formed to penetrate first main surface 101a and second main surface 101b which face each other by their back surfaces. The phrase “face each other by their back surfaces” means that back surfaces of each of first main surface 101a and second main surface 101b are in contact with each other.

A diameter of the predetermined opening is set smaller than a diameter of a cell among cells cultured using cell culture chip 300. Accordingly, fiber mesh sheet 101 has a function of inhibiting passage of cells larger than the predetermined opening from first main surface 101a to second main surface 101b, or from second main surface 101b to first main surface 101a, and a semi-transmissive function of allowing solution components smaller than the predetermined opening (such as test substances or medium components) to pass through.

In addition, fiber mesh sheet 101 has a function as a scaffold for cells to be cultured in cell culture chip 300. Accordingly, for fiber mesh sheet 101, a material that has low toxicity to cells to be cultured and can be bonded thereto may be selected and used.

Fiber mesh sheet 101 is arranged at a position corresponding to the first through-hole and a second through-hole to be described later, and is sandwiched between first partition layer 12 and second partition layer 14 at an outer side of the first through-hole and the second through-hole in a plan view seen from a lamination direction (Z direction).

Accordingly, the first through-hole and the second through-hole are respectively partitioned by fiber mesh sheet 101 at a position at which the first through-hole and the second through-hole overlap. Thereby, first flow path 33, which has first main flow path 36 and is defined by the main surface of first board 11, the first through-hole, and first main surface 101a, is formed. In other words, the main flow path 36 is formed between first board 11 and fiber mesh sheet 101 by the first through-hole.

First main flow path 36 is a part of first flow path 33 formed by the first through-hole. First flow path 33 defined as above is in contact with, in particular, first main flow path 36, and extends in first main flow path 36 along first main flow path 36.

Furthermore, in first flow path 33, first inlet port 34 is formed at one end corresponding to hole 31, and first outlet port 38 is formed at the other end, and they respectively communicate with the outside of cell culture chip 300 through holes 31.

Furthermore, first flow path 33 includes first inlet flow path 35 leading to first main flow path 36 from first inlet port 34, and first outlet flow path 37 leading to first main flow path 36 from first outlet port 38. First inlet flow path 35 and first outlet flow path 37 are defined from first main flow path 36 by second partition layer 14 instead of fiber mesh sheet 101.

### Second Partition Layer

Second partition layer 14 is a plate-shaped member formed of a silicone resin. Details will be described later, but the second through-hole corresponds to second flow path 41. Both ends of the second through-hole correspond to hole 31 formed in first board 11, and hole 32 formed in first partition layer 12.

### Second Board

Second board 15 is a plate-shaped member formed of a material such as glass. A material of second board 15 is not limited to glass, and any material such as resin and ceramics may be used. In the first exemplary embodiment, second board 15 is a plate-shaped member having a rectangular main surface.

Thereby, second flow path 41, which has second main flow path 44 and is defined by the main surface of second board 15, the second through-hole, and second main surface 101b, is formed.

In other words, second main flow path 44 is formed between second board 15 and fiber mesh sheet 101 by the second through-hole. Second main flow path 44 is a part of second flow path 41 formed by the second through-hole.

Fiber mesh sheet 101 is disposed between first flow path 33 and second flow path 41 such that first main flow path 36 of first flow path 33 is located on first main surface 101a, and second main flow path 44 of second flow path 41 is located on second main surface 101b.

Accordingly, via fiber mesh sheet 101, first main flow path 36 and second main flow path 44 can exchange a component smaller than a predetermined hole diameter, such as a test substance and a medium component flowing through the respective flow paths.

Furthermore, in second flow path 41, second inlet port 42 is formed at one end corresponding to hole 31 and hole 32, and second outlet port 46 is formed at the other end, and they respectively communicate with the outside of cell culture chip 300 through holes 31 and holes 32.

Furthermore, second flow path 41 includes second inlet flow path 43 leading to second main flow path 44 from second inlet port 42, and second outlet flow path 45 leading to second main flow path 44 from second outlet port 46. Second inlet flow path 43 and second outlet flow path 45 are defined from second main flow path 44 by first partition layer 12 instead of fiber mesh sheet 101.

That is, in a plan view seen from the lamination direction (Z direction), first inlet flow path 35 and second inlet flow path 43 do not overlap, and first outlet flow path 37 and second outlet flow path 45 do not overlap. Accordingly, first inlet flow path 35 and first outlet flow path 37 form a part of first flow path 33 by the main surface of second partition layer 14 on which the second through-hole is not formed.

Furthermore, second inlet flow path 43 and second outlet flow path 45 form a part of second flow path 41 by the main surface of first partition layer 12 on which the first through-hole is not formed.

### Comparison Between Examples and Comparative Examples

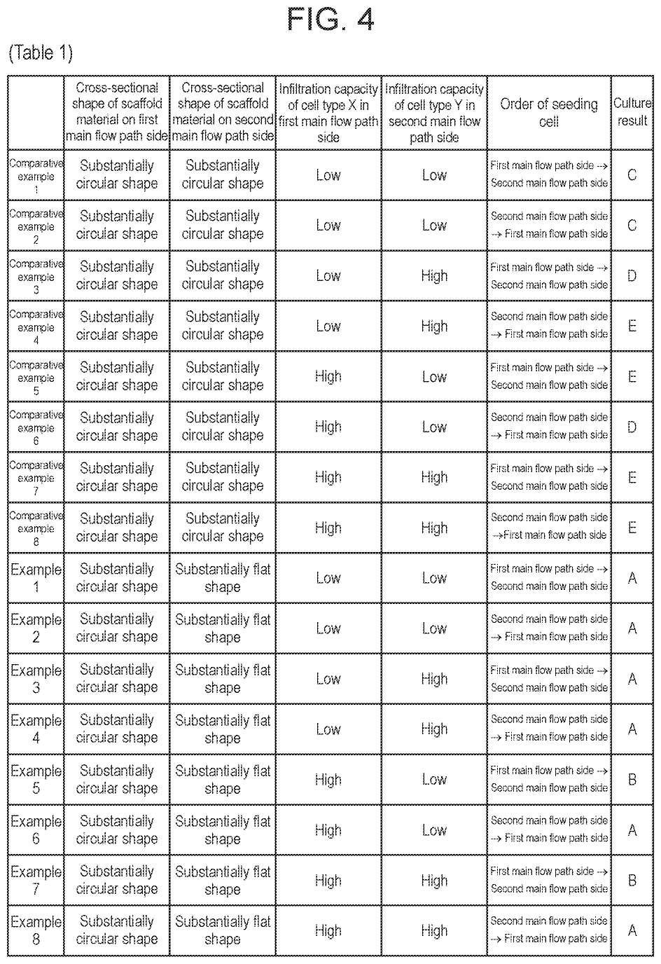

Hereinafter, comparative examples and examples in the first exemplary embodiment will be described with reference to Table 1 of FIG. 4.

For both of the comparative examples and the examples, fiber mesh sheets were produced by the method for manufacturing a fiber mesh sheet described in the first exemplary embodiment. The fiber mesh sheet of a first layer was installed on a second main flow path side, and the fiber mesh sheet of a second layer was installed toward a first main flow path with respect to the cell culture chip described in the first exemplary embodiment.

In the comparative examples, although intertwining between the first layer and the second layer was formed by controlling a heating temperature/time in S05 in the method for manufacturing a fiber mesh sheet according to the present exemplary embodiment, fibers each having a substantially circular shape for a cross-sectional shape were used for both of the first layer and the second layer.

Furthermore, as cells to be seeded in the cell culture chip, cells having low infiltration capacity and cells having high infiltration capacity were used as a cell type X to be seeded on a first main flow path side, and cells having low infiltration capacity and cells having high infiltration capacity were used as a cell type Y to be seeded on a second main flow path side. Culture was evaluated by changing the order of seeding on the first main flow path side and the second main flow path side.

In the exemplary embodiment, for both of the cell type X and the cell type Y, a cell having one cell size equivalent to 20 μm was used, and a polymer compound solution of 1 μM (molecular weight equivalent to 750,000) was used as a test substance.

The fiber mesh sheet produced by the method for method for manufacturing a fiber mesh sheet according to the present exemplary embodiment had a fiber having a diameter equivalent to 5 μm, and an opening that is equivalent to 25 μm and is around the fiber at intervals equivalent to 30 μm, which was larger than a size equivalent to 20 μm that is a size of one cell. Accordingly, the polymer compound solution 1 μM (equivalent to a molecular weight of 750,000) which was used as a test substance was not easily clogged.

Meanwhile, evaluation results of culture show a state in which the cell type X and the cell type Y were respectively formed in a sheet form without being mixed to each other, and A to E are as follows.

A: A rate at which the cell type X and the cell type Y were respectively formed in a sheet form without being mixed to each other was 100%.

B: A rate at which the cell type X and the cell type Y were respectively formed in a sheet form without being mixed to each other was less than 100% and equal to or more than 80%.

C: A rate at which the cell type X and the cell type Y were respectively formed in a sheet form without being mixed to each other was less than 80% and equal to or more than 60%.

D: A rate at which the cell type X and the cell type Y were respectively formed in a sheet form without being mixed to each other was less than 60% and equal to or more than 40%.

E: A rate at which the cell type X and the cell type Y were respectively formed in a sheet form without being mixed to each other was less than 40%.

In Comparative Example 1 and Comparative Example 2 in Table 1, in a case where cells having low infiltration capacity were used for both of the cell type X seeded on the first main flow path side and the cell type Y seeded on the second main flow path side, a culture result was C regardless of the order of seeding the cells.

It is presumed that the reason for this is because an opening size of the fiber mesh sheet was larger than a size of one cell to be seeded, and thereby some cells slipped from the main flow path side at which the cells were seeded to the opposite main flow path side.

On the other hand, in Comparative Example 3 and Comparative Example 4, in a case where cells having high infiltration capacity were used for the cell type Y seeded on the second main flow path side, a culture result of the case in which the order of seeding the cells was from the first main flow path side to the second main flow path side was D, which was a deteriorated result, and a culture result of the case in which the order of seeding the cells was from the second main flow path side to the first main flow path side was E, which was a further deteriorated result.

It is presumed that the reason for this is because, in Comparative Example 3, although most of the cells having low infiltration capacity and seeded on the first main flow path side were first formed in a sheet form, most of the cells having high infiltration capacity and subsequently seeded on the second main flow path side slipped toward the first main flow path side and mixed with the above cells.

Furthermore, it is presumed that in Comparative Example 4, almost all the cells having high infiltration capacity and seeded on the second main flow path side slipped from the fiber mesh sheet toward the first main flow path side, and therefore the cell type X subsequently seeded on the first main flow path side could not be formed in a sheet form.

Similarly, in Comparative Example 5 and Comparative Example 6, in a case where the cell type X seeded on the first main flow path side was used for cells having high infiltration capacity, and the cell type Y seeded on the second main flow path side was used for cells having low infiltration capacity, a result of Comparative Example 5 in which the cells having high infiltration capacity were seeded first was E, and a result of Comparative Example 6 in which the cells having high infiltration capacity were subsequently seeded was D, as in the above-described cases of Comparative Example 3 and Comparative Example 4.

Meanwhile, in Comparative Example 7 and Comparative Example 8, in a case where cells having high infiltration capacity were used for both of the cell type X seeded on the first main flow path side and the cell type Y seeded on the second main flow path side, a culture result was E regardless of the order of seeding the cells. Also in this case, it is presumed that cells seeded first slipped from the fiber mesh sheet toward the main flow path side at which the cells were to be seeded subsequently, and thereby a proportion of formation of the cell type seeded subsequently in a sheet form was minimized.

Meanwhile, the culture results were improved in all the examples corresponding to the comparative examples, and this tendency was particularly remarkable in the examples in which the cell type having high infiltration capacity was seeded first from the second main flow path side (Examples 4, 6, and 8).

That is, it is presumed that, by firstly seeding the cell type Y having high infiltration capacity from the second main flow path side using fiber mesh sheet 101 of the exemplary embodiment, the cell type Y was cultured in a sheet form on the second main flow path side without slipping from fiber mesh sheet 101, and the cell type X seeded on the first main flow path side was subsequently cultured in a sheet form regardless of whether infiltration capacity was high or low, and thereby all cells of the cell types X and Y on both the first main flow path side and the second main flow path side could be laminated in a sheet form.

This means that the cell type having high infiltration capacity is likely to slip from a side having a substantially circular shape, but is unlikely to slip from a side having a substantially flat shape.

That is, it is presumed that the cell type having high infiltration capacity is likely to fit into gaps of the mesh by being wet in a lateral direction rather than on a surface having a substantially circular shape, and thereby is likely to slip from openings, whereas as compared to the side having a substantially circular shape, the cell type is likely to get wet in a surface direction in the case of the side having a substantially flat shape, and thereby a phenomenon of fitting into gaps of the mesh is inhibited.

In addition, in a case where the cell type X having high infiltration capacity was seeded on the first main flow path side, it can be further expected that a cell sheet having a function closer to that of a living body can be obtained by wetting in a lateral direction of the substantially circular shape to bring water into contact with the cell type Y seeded on the second main flow path side in advance through a mesh opening.

In order to obtain such an effect, in the exemplary embodiment, the most desirable contact angle between the substantially circular shape and water was set to 90°, but it is expected that the same effect would be exhibited in a case where a contact angle is set to 90° or more and 150° or less.

Furthermore, in the exemplary embodiment, the cell type X and the cell type Y each having one cell size equivalent to 30 μm were used, 1 μM (molecular weight equivalent to 750,000) was used as a test substance, and a gap of fiber mesh sheet 101 was set to 30 μm that enabled inhibition of clogging with a test substance, but these values can be appropriately set.

Specifically, as a lower limit, it is required to set a space to be larger than a size at which a test substance to be used is clogged depending on molecular weights and concentrations of the test substance, but as an upper limit, it is sufficient for a space to be a space that can inhibit passage of seeded cells.

Furthermore, in the exemplary embodiment, a polystyrene fiber having a diameter of 5 μm was used, but a diameter can be set as appropriate.

Specifically, a diameter may be 1 μm or more and 50 μm or less, which is expected to function as a scaffold for cells to be cultured. A material is not limited to polystyrene, and it may be polylactic acid-based materials or silicone-based materials having low cytotoxicity, but it is desirable to use a polymer material because it is required to have flexibility as a function of a scaffold of cells.

In addition, an in-plane rotation angle of 90° in the step S03 of the method for manufacturing a fiber mesh sheet according to the present exemplary embodiment is not limited to this angle, and when an angle is 30° or more and 150° or less, it is possible to inhibit both passage of cells and clogging with a test substance.

In addition, in a case where it is required to increase a thickness of the fiber mesh sheet depending on cell culture chips to be used, it is sufficient for rotation and spinning of a film to be repeated in the same manner after the step S04 of the method for manufacturing a fiber mesh sheet described in the present exemplary embodiment. In this case, it is desirable to unify rotation angles and spaces between fibers in order to inhibit clogging with a test substance.

### Capturing and Recovering at One Cell Level

The effects shown by the fiber mesh sheet of the exemplary embodiment in the cell culture chip are also effective for capturing and recovering cells.

Specifically, for example, in a case where the fiber mesh sheet is used as a separation diaphragm for capturing and recovering a substance having a size of one cell (for example, a red blood cell having a diameter equivalent to 8 μm) from a sample (for example, blood), in the comparative example using only fibers having a substantially circular shape for a cross-sectional shape, by wetting in a lateral direction than a surface having a substantially circular shape, the substance having the size of one cell is likely to fit into gaps of the mesh, and thereby it is difficult to recover the substance although it can be captured.

Meanwhile, in the example using fibers having a substantially flat shape for a cross-sectional shape, it becomes easier to wet in a surface direction as compared with the substantially circular shape, and thereby it is possible to inhibit a phenomenon of fitting into gaps of the mesh, and to achieve both capturing and recovering of a substance having a size of one cell.

The present disclosure includes an appropriate combination of any exemplary embodiment and/or example among the various exemplary embodiments and/or examples described above, and the effects of each of the exemplary embodiments and/or examples can still be exhibited.

According to the fiber mesh sheet according to the present disclosure, use of the fiber mesh sheet contributes to, for example, new expansions in development of pharmaceutical products and the like, such as establishment of a test system by cells cultured using a cell culture chip.

What is claimed is:

1. A fiber mesh sheet,
wherein the fiber mesh sheet has a mesh structure in which two or more layers of planar fiber arrangement groups are laminated, where, in each of the planar fiber arrangement groups, longitudinal directions of a plurality of fibers made of a polymer material are arranged in a plane along one direction,
longitudinal directions of the fibers in one of the fiber arrangement groups intersect those of the fibers in the other fiber arrangement group in two adjacent layers of the fiber arrangement groups at an intersecting angle of 30° or more and 150° or less in a plan view seen from a direction perpendicular to the plane,
an upper part of a cross section of the fiber in the fiber arrangement group at a lowermost layer is a substantially circular shape, and a lower part of a cross section of the fiber in the fiber arrangement group at the lowermost layer is a substantially flat shape, where the upper part is a side on which the adjacent fiber arrangement group is present, and the lower part is a side on which the adjacent fiber arrangement group is not present, and
a cross section of the fiber in the fiber arrangement group at a layer other than the lowermost layer is a substantially circular shape.

2. The fiber mesh sheet of claim 1,
wherein a contact angle between the substantially circular shape in the fiber arrangement group at the layer other than the lowermost layer and water is 90° or more and 150° or less.

3. The fiber mesh sheet of claim 1,
wherein an average diameter of the fibers in the fiber arrangement group is 1 μm or more and 50 μm or less.

4. A method for manufacturing a fiber mesh sheet, the method comprising:
a step of laminating two or more layers of planar fiber arrangement groups, where, in each of the planar fiber arrangement groups, longitudinal directions of a plurality of fibers made of a polymer material are arranged in a plane along one direction, such that longitudinal directions of the fibers in one of the fiber arrangement groups intersect those of the fibers in the other fiber arrangement group in two adjacent layers of the fiber arrangement groups at an intersecting angle of 30° or more and 150° or less in a plan view seen from a direction perpendicular to the plane; and
a step of performing a heat treatment at a temperature equal to or higher than a melting point of the polymer material of the fiber and lower than a temperature that the fiber is melted and cut,
wherein the two adjacent layers of the fiber arrangement groups are intertwined by the step of performing the heat treatment at a majority of portions at which the two adjacent layers are in contact with each other.

5. The method for manufacturing a fiber mesh sheet of claim 4,
wherein a lower part of a cross section of the fiber in the fiber arrangement group at a lowermost layer is formed into a substantially flat shape by the step of performing the heat treatment at the temperature equal to or higher than the melting point of the polymer material of the fiber and lower than the temperature that the fiber is melted and cut, where the lower part is a side on which the adjacent fiber arrangement group is not present.

6. A cell culture chip comprising the fiber mesh sheet of claim 1.
